(19) United States
(12) Patent Application Publication
(10) Pub. No.: US 2008/0077212 A1
(43) Pub. Date: Mar. 27, 2008

(54) Hypothermia Warming System

(76) Inventor: David Hammac, Norwood, NY (US)

Correspondence Address: WILSON DANIEL SWAYZE, JR., 3804 CLEARWATER CT., PLANO, TX, 75025, US

(21) Appl. No.: 11/534,396

(22) Filed: Sep. 22, 2006

(51) Int. Cl. A61F 7/00

(52) U.S. Cl. 607/108; 607/112

(57) ABSTRACT

A system for warming of a user includes the first torso warming device to warm the torso of a user, the first torso warming device including a first strap to connect the torso warming device to the user.

### FIELD OF THE INVENTION

The present invention relates to warming devices and more particularly to electrically heated, temperature controlled devices for medical applications, warming specific areas of the body to prevent or treat hypothermia by increasing the body temperature.

### BACKGROUND OF THE INVENTION

Hypothermia is a condition where the body temperature of a person falls below normal, usually as a result of exposure to low temperatures. For example, a person may become lost injured or otherwise incapacitated while outside during winter or at night when the temperature falls, and the person is unprepared for the colder temperatures.

A person who is exposed to cold water as a result of falling through ice, a boating accident or other situations can quickly reach dangerously low temperatures if untreated. Hypothermia can result in frostbite, cardiac arrest, and eventually death.

The present treatment for hypothermia includes providing warm humid air for the person to breathe, blankets and rapid core rewarming. Core temperature after drop is one of the most significant causes of arrhythmia in the hypothermic patient. However, the heat generating equipment to treat these conditions may not be available especially if the patient is in an isolated area. Furthermore, if the heating equipment is costly, heavy or bulky, the heating equipment may not be immediately available, requiring a large amount of time for the heating equipment to be transported to the location where it is required.

As a consequence, conventional external warming techniques provide various challenges to the rescuer. Electric blankets warm too much of the skin surface of the patient which results in core temperature after drop. The electric vests and jackets may be difficult to apply to unstable patients especially if cervical/spinal injury is suspected. Furthermore jackets interfere with blood-pressure assessment and intravenous therapy. The above described devices could warm too much of the body surface for severely hypothermic patients, increasing the risk of core temperature after drop. Additionally, jackets and vests should be properly sized in order to be effective. Since the size of the patient cannot be predicted, various sizes would have to be carried.

Active internal warming is the best solution for the severely hypothermic patients, but these procedures are too difficult to perform in a prehospital setting. These procedures require complex equipment, highly trained medical personnel and a controlled environment to perform this type of active core warming.

Examples of torso warmers and warming devices are described in U.S. Pat. Nos. 6,439,942, 3,999,037, 6,049,062, 5,977,517, 5,893,991, 4,061,897, 4,404,460, 5,032,705, 6,138,664, 6,185,744, 6,329,638, 6,550,471, 6,649,873, and 6,770,848.

Another example of a torso warmer is found in my application Ser. No. 10/709,070 which was filed on Apr. 10, 2004 and is incorporated by reference in its entirety and disclosure document 593867 and 595432.

### SUMMARY

The present invention describes a portable heating system for providing warmth to a user at specific areas of the body. Placement of the warming devices of the present invention allows extremities and other skin areas to warm at a slower rate which reduces the risk of core temperature after drop.

The warming device of the present invention is suitable for warming people who are suffering from hypothermia and who may be injured, lying down, unconscious or cannot be safely moved without assistance from medically trained personnel.

The warming device of the present invention can be applied to patients without the need for these patients to be moved excessively.

The warming device of the present invention can operate off a DC battery, preferably 12 V.

The warming device of the present invention can operate off a 12 V service outlet which is available on most vehicles.

The warming devices of the present invention can operate off household current through the use of the power adapter.

The warming devices of the present invention can operate off all of the above described devices individually or in combination.

The warming devices of the present invention are easy to use, dependable and temperature controllable for patient safety.

The present invention includes a torso warming device which can be used as a sole external warming device to concentrate heat around the victims upper torso providing heat to the axillary blood vessels and chest wall. This allows the lower torso and extremities to warm at a slower rate reducing the risk of core temperature after drop

The present invention includes a non-constrictive neck collar which can be used to supplement the upper torso warming device to warm the carotid/blood vessels and trachea.

The present invention includes a groin pad that can be applied along with the torso warming device and neck collar.

A second torso warming device can be applied to the lower torso by rotating the second torso warming device 180° and attaching it to the first torso warming device.

The present invention is useful in outdoor recreation, search and rescue, maritime, military, ambulances and hospitals.

Any combination of the above warming devices can accomplish the objectives of the present invention

The present invention does not require arm openings or leg openings through which a person's arms or legs are inserted.

The warming devices of the present invention warm the important external areas of the body and allows the extremities to warm at a slower rate which will reduce the risk of core temperature after drop.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a cross-sectional view of the neck collar of the present invention;

FIG. 2 illustrates a front view of the neck collar electrical heating wire diagram of the present invention;

FIG. 3 illustrates the neck collar with the protective covering of the present invention;

FIG. 4 illustrates the neck collar and patient barrier covering of the present invention;

FIG. 5 illustrates a groin warming pad heating wire diagram of the present invention;

FIG. 6 illustrates another groin warming pad wire diagram of the present invention;

FIG. 7 illustrates the external side of the groin warming pad of the present invention;

FIG. 8 illustrates the interior side of the groin warming pad of the present invention;

FIG. 9 illustrates the another interior side of the groin warming pad;

FIG. 10 illustrates the groin warming pad which has been positioned on the patient;

FIG. 11 illustrates the axillary warming pad of the present invention;

FIG. 12 illustrates the axillary warming pad being applied to the patient;

FIG. 13 illustrates a first configuration for the heater wire of the present invention;

FIG. 14 illustrates a second configuration for the heater wire of the present invention;

FIG. 15 illustrates a cross-sectional view of the axillary warming pad;

FIG. 16 illustrates a fastening device of the present invention;

FIG. 17 illustrates an external view of the upper torso warming device;

FIG. 18 illustrates an interior view of the upper torso warming device;

FIG. 19 illustrates a cross-sectional view of the upper torso warming devices wiring diagram;

FIG. 20 illustrates a first torso warming device and a second torso warming device attached to one another;

FIG. 21 illustrates a power module for providing electrical power to the warming devices of the present invention;

FIG. 22 illustrates a configuration for the power module of the present invention;

FIG. 23 illustrates a patient using the warming devices of the present invention;

FIG. 24 illustrates a alternate apparatus to prevent or treat hypothermia in accordance with the teachings of the present invention;

FIG. 25 illustrates a patient using the warming device of the present invention.

### DETAILED DESCRIPTION

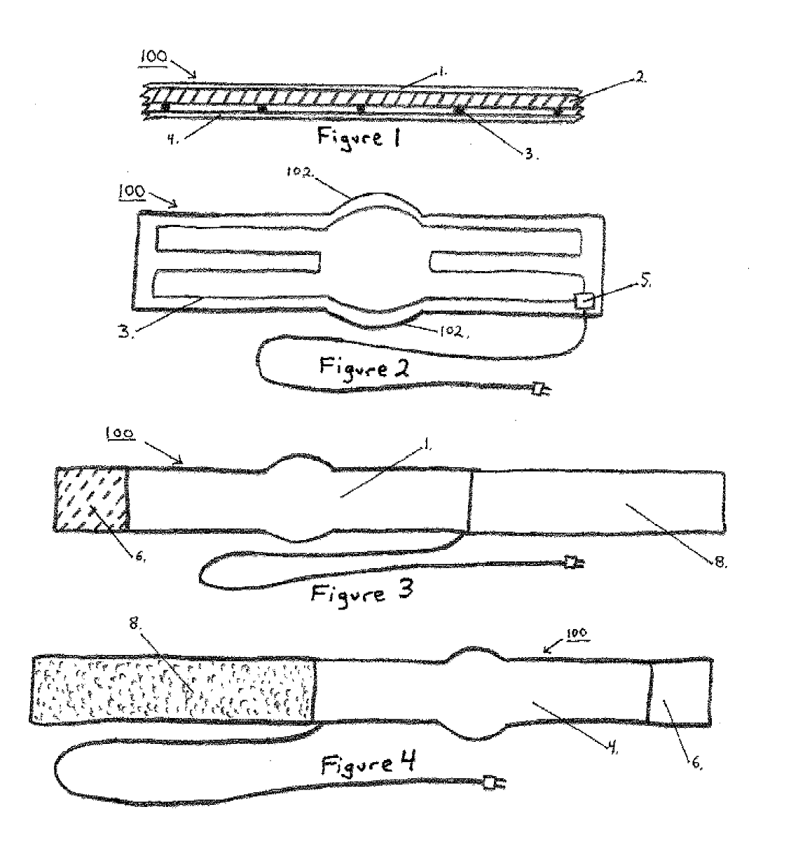

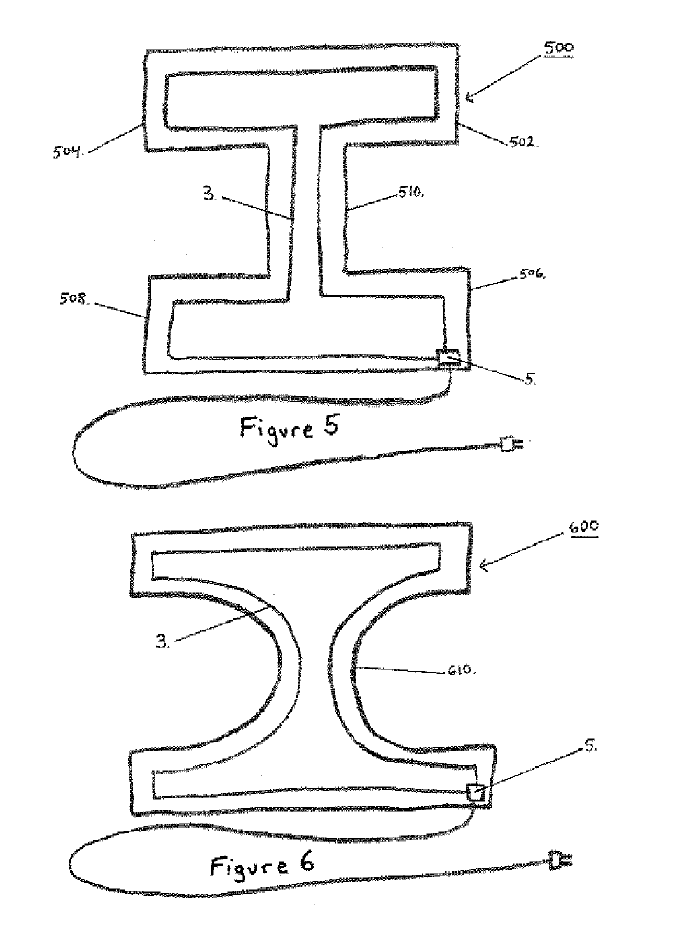

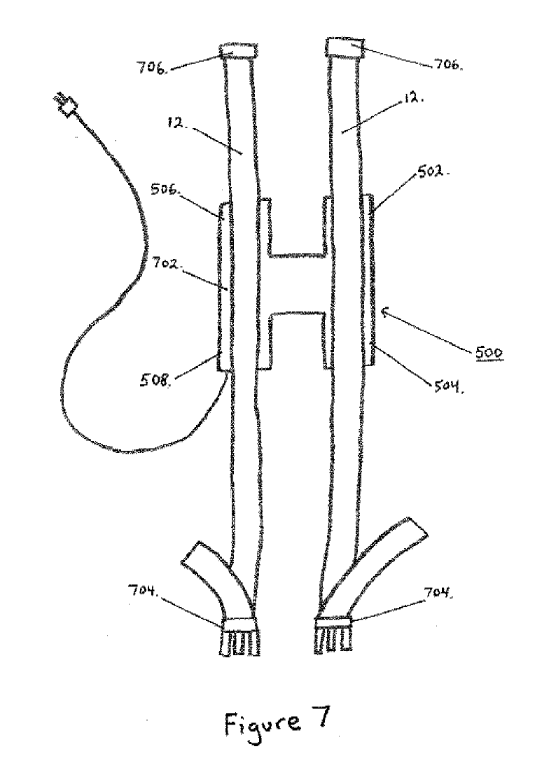

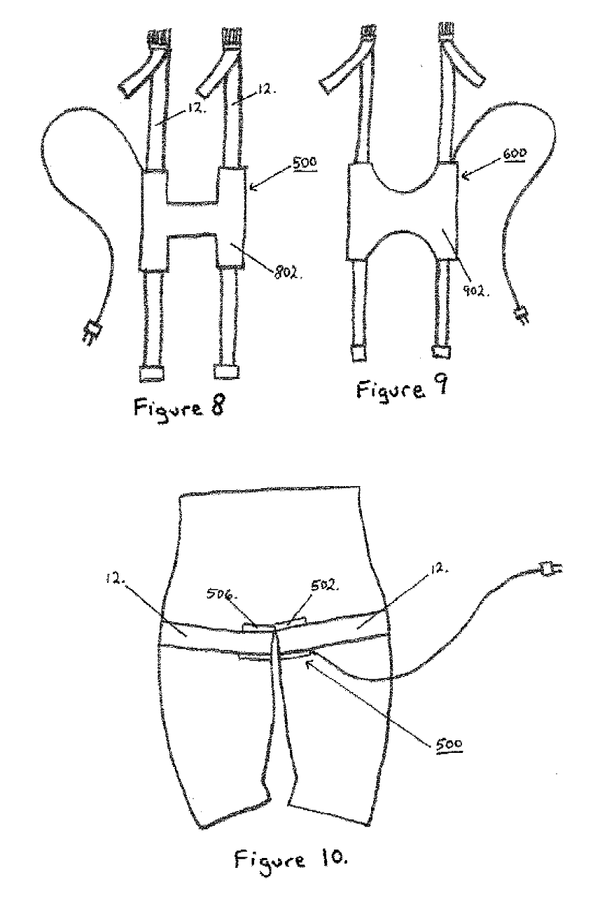

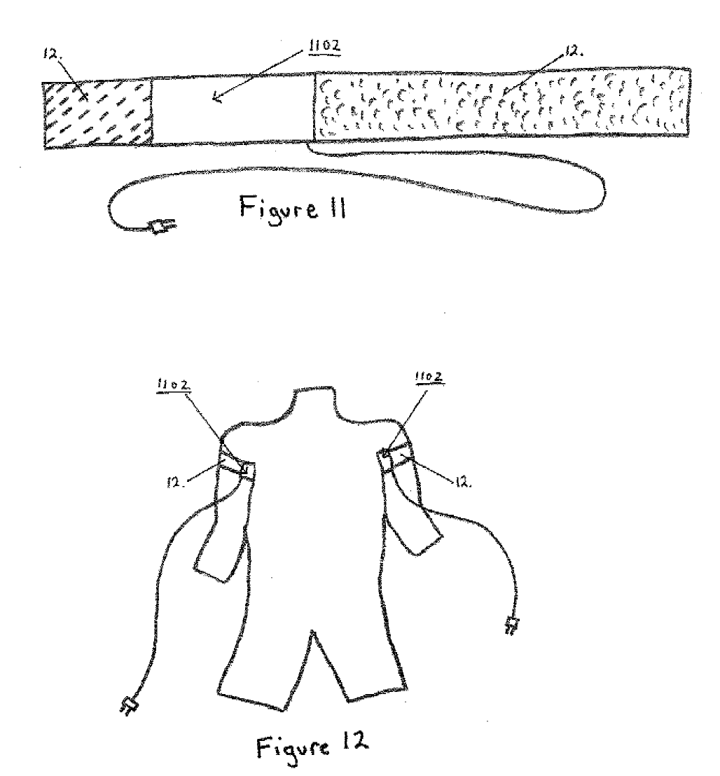

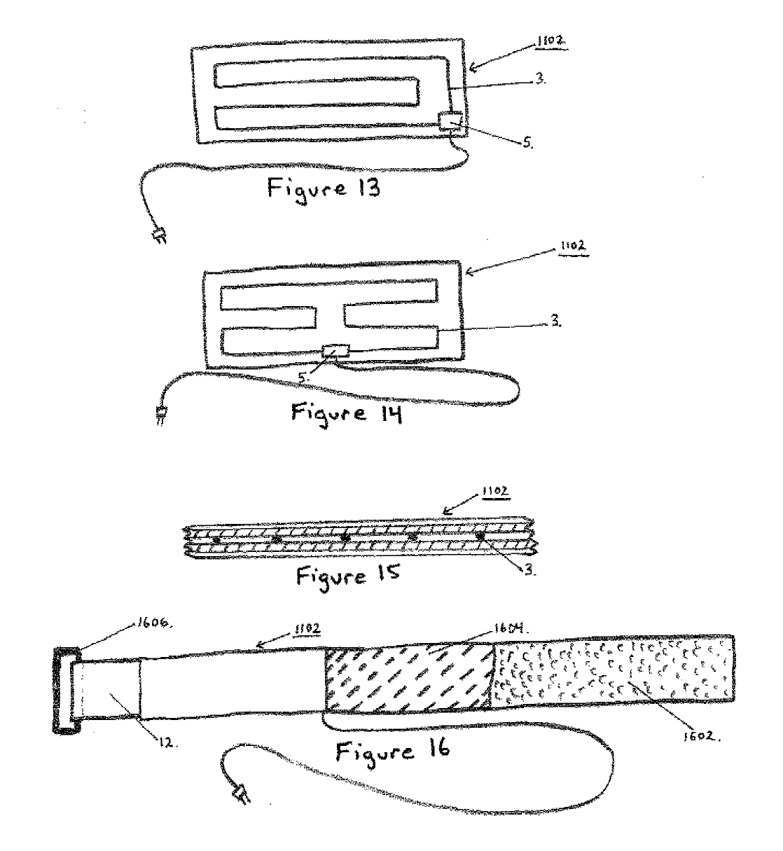

FIG. 1 illustrates a cross-sectional view of a neck collar 100 to provide heat to only the neck of the patient. The neck collar 100 includes a protective covering 1 to protect the neck collar 100 from the heating elements. The protective covering 1 may be formed from flexible and/or durable material such as nylon, vinyl, cotton, canvas or other suitable material. The neck collar 100 additionally includes a layer of insulation 2 to reflect heat back to the patient, heater wire 3 which is resistive in order to convert the electricity to heat for the patient and patient barrier covering 4 to protect the skin of the patient from direct contact with the heater wire 3 and may be formed from fleece, cotton or other suitable material.

FIG. 2 illustrates a front view of the neck collar 100 of the present invention. The neck collar 100 is substantially rectangular with a pair of raised portions 102 to be placed under the chin of the patient. FIG. 2 shows a path for the heater wire 3, and FIG. 2 shows the heater wire 3 connected to a thermistor 5 to measure the temperature and regulate the temperature by connecting and disconnecting the electricity to the heater wire 3.

FIG. 3 illustrates the neck collar 100 showing the protective covering 1 in approximately the center of the neck collar 100 and a first fastening device 6 and a second fastening device 8 on opposing ends of the neck collar 100. The first fastening device 6 and a second fastening device 8 may be the hooks and loops of Velcro or buttons, buckles, magnetic fasteners or any other suitable device.

FIG. 4 illustrates the neck collar 100 showing the patient barrier covering 4 in approximately the center of the neck collar 100 and the first fastening device 6 and the second fastening device 8 on reversed opposing ends of the neck collar 100.

FIG. 5 illustrates a groin/femoral warming pad 500 which is shown having an H shape to warm only a portion of the groin. The groin warming pad 500 includes a first arm 502, a second arm 504, a third arm 506, a fourth arm 508 and a connecting member 510. The connecting member 510 is substantially perpendicular to the first arm/flap 502, the second arm/flap 504, the third arm/flap 506 and the fourth arm/flap 508. FIG. 5 additionally illustrates a path for the heater wire 3 and the thermistor 5.

FIG. 6 illustrates an alternate design for the groin warming pad 600 including the connecting member 610 which is convexly shaped. FIG. 6 additionally illustrates the path for the heater wire 3 which is connected to the thermistor 5.

FIG. 7 illustrates the external side of the groin warming pad 500 and shows the first strap 12 connected to the first arm 502 and the second arm 504 and the second strap 12 connected to the third arm 506 and the fourth arm 508. The strap 12 may be formed from nylon, cotton or any other suitable material. The exterior surface 702 of the groin warming pad 500 may be formed from nylon, cotton, canvas, vinyl or any other suitable material. FIG. 7 additionally illustrates a fastening device 704, 706 at opposing ends of the strap 12.

FIG. 8 illustrates the interior side 802 of the groin warming pad 500 which is placed against the patient and shows the straps 12.

FIG. 9 illustrates the interior side 902 of the groin warming pad 600 which is placed against the patient and shows the straps 12.

FIG. 10 illustrates the groin warming pad 500 which has been positioned on the patient. The groin warming pad 500 is held in place by the strap 12 which extends around the upper leg of the patient. FIG. 10 shows the first arm 502 and the third arm 506 being held in the groin of the patient.

FIG. 11 illustrates an axillary warming pad 1102 to produce heat on the external side of the axillary warming pad 1102 and to produce heat on the interior side of the axillary warming pad 1102 to warm only a portion of the armpit. The axillary warming pad 1102 could be placed around the upper arms of the patient at the armpit in order to take advantage of the heating on the interior side and the heating on the external side. FIG. 11 illustrates that the axillary warming pad 1102 includes straps 12 on opposing ends of the axillary warming pad 1102. The straps 12 include a fastening device such as Velcro, buckles, buttons and other appropriate devices.

FIG. 12 illustrates the axillary warming pad 1102 being applied to the upper arms of the patient at the armpit. The straps 12 hold the axillary warming pad 1102 in position on the upper arms of the patient at the armpit.

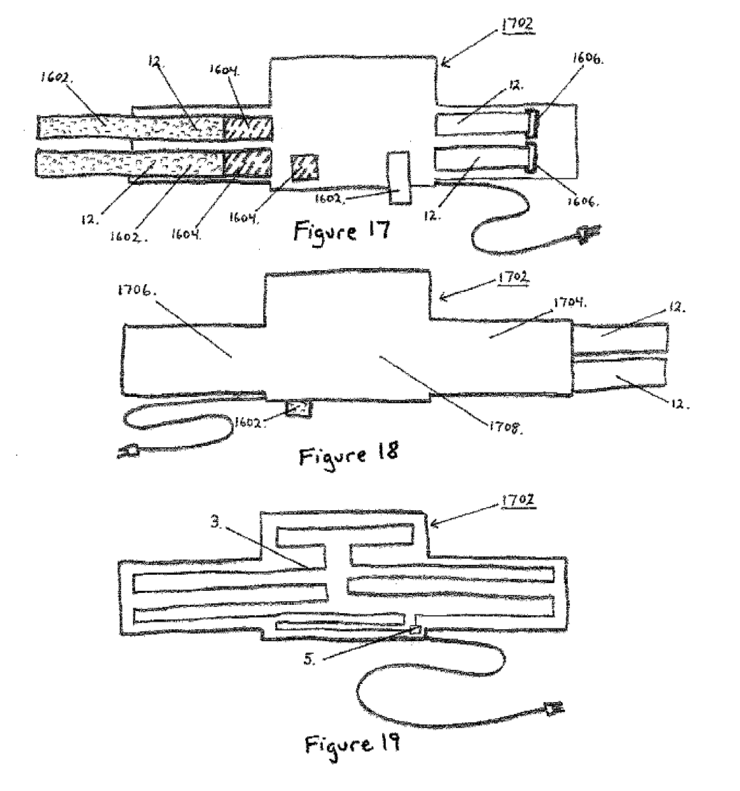

FIG. 13 illustrates a first configuration for the heater wire 3 of the axillary warming pad 1102. The heater wire 3 forms an approximate U-shape and is connected to the thermistor 5.

FIG. 14 illustrates a second configuration for the heater wire 3 of the axillary warming pad 1102. The heater wire 3 is shown as being H shaped.

FIG. 15 illustrates a cross-sectional view of the axillary warming pad 1102 to apply heat to the external and internal side of the axillary warming pad 1102.

FIG. 16 illustrates an additional fastening device 1602, 1604, 1606 connected to the strap 12. The fastening device 1606 is shown as a buckle to connect to the first Velcro section 1602 and the second Velcro section 1604 on the opposing end of the axillary warming device 1102. The first Velcro section 1602 and the second Velcro section 1604 are adjacent, and one of the first and second Velcro section 1602, 1604 includes the hooks of Velcro and the other of the first and second Velcro section 1602, 1604 includes the loops of Velcro. The buckle 1606 is positioned approximately at the junction of the first Velcro section 1602 and the second Velcro section 1604, and the first and second Velcro section 1602, 1604 are pressed together to engage the hoops and loops of Velcro.

FIG. 17 illustrates an external view of the torso warming device 1702 to warm only a portion of the torso for example only the upper torso and which includes a fastening device which is shown as the first Velcro section 1602 and the second Velcro section 1604 for each strap. FIG. 17 illustrates two straps 12 which are connected to two buckles 1606. Any number of straps 12 could be incorporated with the teachings of the present invention. The first Velcro section 1602, the second Velcro section 1604 and the buckle 1606 function as described in FIG. 16. FIG. 17 illustrates that the exterior surface of the torso warming device 1702 includes a first Velcro section 1602 and a second Velcro section 1604 for connection to an other torso warming device (not shown).

FIG. 18 illustrates an interior view (patient side) of the torso warming device 1702 which includes a first upper torso arm 1704 and a second torso arm 1706 which extends from a central section 1708 of the upper torso warming device. FIG. 18 additionally illustrates the straps 12 extending from the first upper torso arm 1704.

FIG. 19 illustrates a cross-sectional view of the torso warming device 1702 which shows a path of heater wire 3 which is connected to a thermistor 5. Other paths of heater wire 3 are within the scope of the present invention.

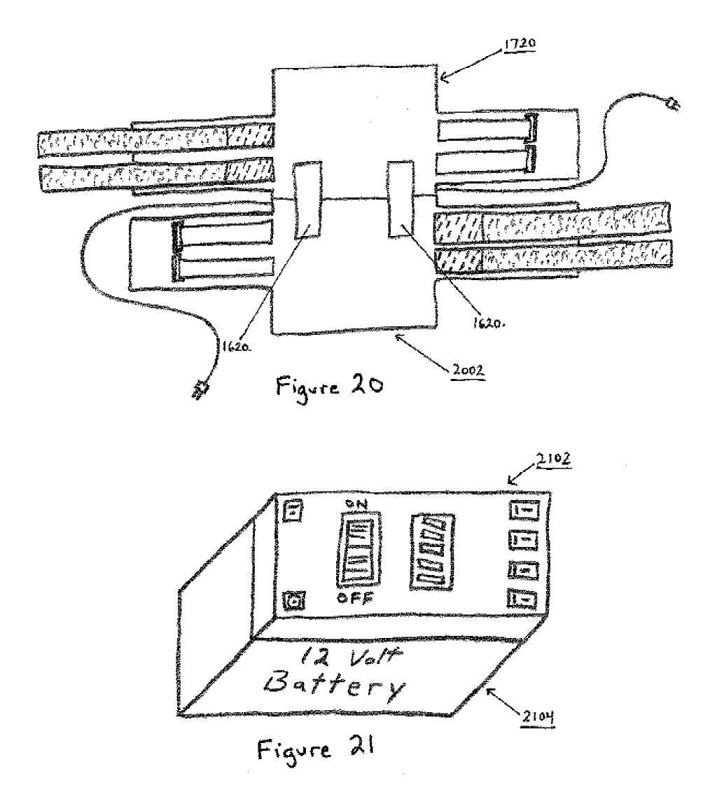

FIG. 20 illustrates a first torso warming device 1720 connected to a second torso warming device 2002. One way of achieving the result illustrated in FIG. 20 is to rotate the torso warming device 1720 by 180°. The second torso warming device 2002 can be used to warm the lower torso of the patient. The second torso warming device 2002 is connected to the first torso warming device 1720 by the fastening device 1620 which is shown in FIG. 20 as Velcro.

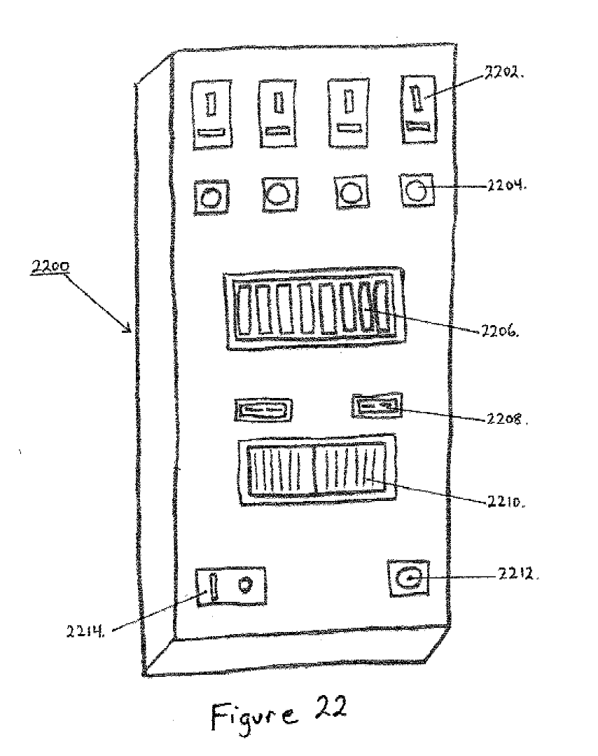

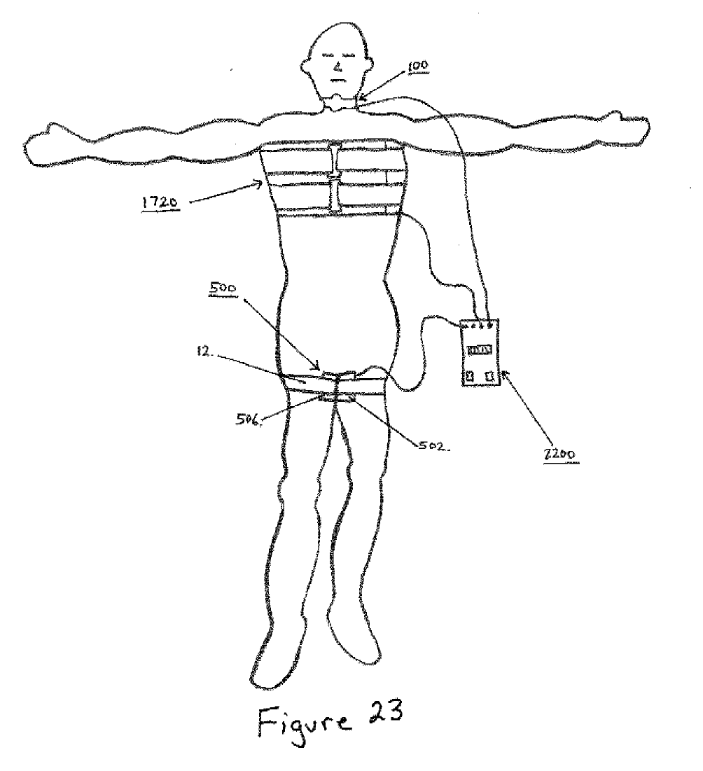

FIG. 21 illustrates a power module 2102 for providing electrical power to the above described warming devices which may be attached to a 12 V sealed lead acid battery 2104 to provide portable power.

FIG. 22 illustrates a potential configuration for the power module 2200. The power module 2200 includes power outlet 2202 for connecting to the warming devices described in the present application, trip fuses 2204 to protect the circuits, a power level display 2206 to display the power level, a battery fuse 2208, a switch 2210 to switch between external power supply, internal power supply and off. Additionally the power module 2200 includes an AC to DC recharge outlet 2212 and an external power supply and outlet 2214.

FIG. 23 illustrates a patient laying supine with a torso warmer 1720 applying warmth to the chest wall and penetrating to heat the vascular lung tissue and axillary blood vessels. The neck collar 100 is applied loosely to the neck of the patient in order to provide warmth to the carotid blood vessels and trachea. The groin warming pad 500 provides warmth to the femoral blood vessels. The configuration described above could be used to prevent or treat mild, moderate and severe hypothermia with important external areas of the body being warmed. The majority of the body surfaces are correspondingly left to be warmed at a slower rate reducing the risk of core temperature after drop.

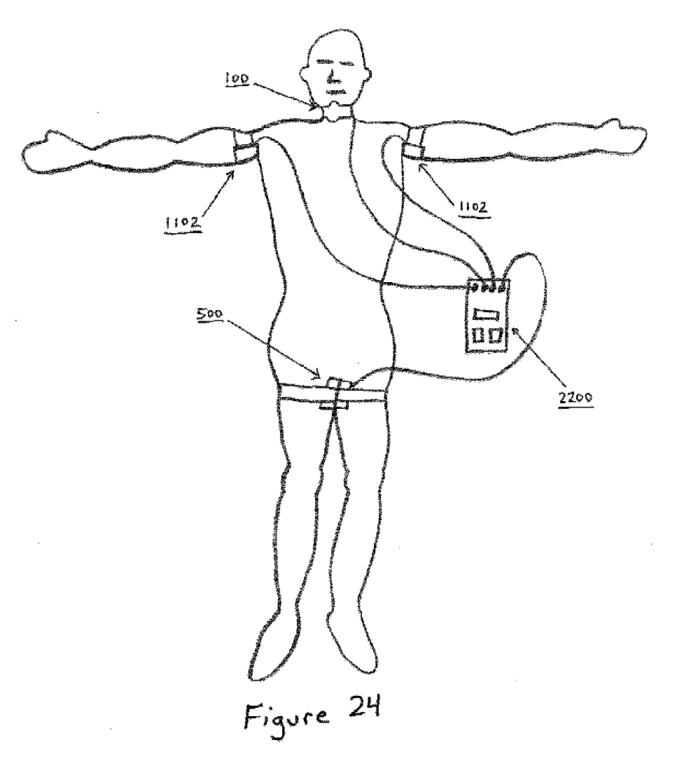

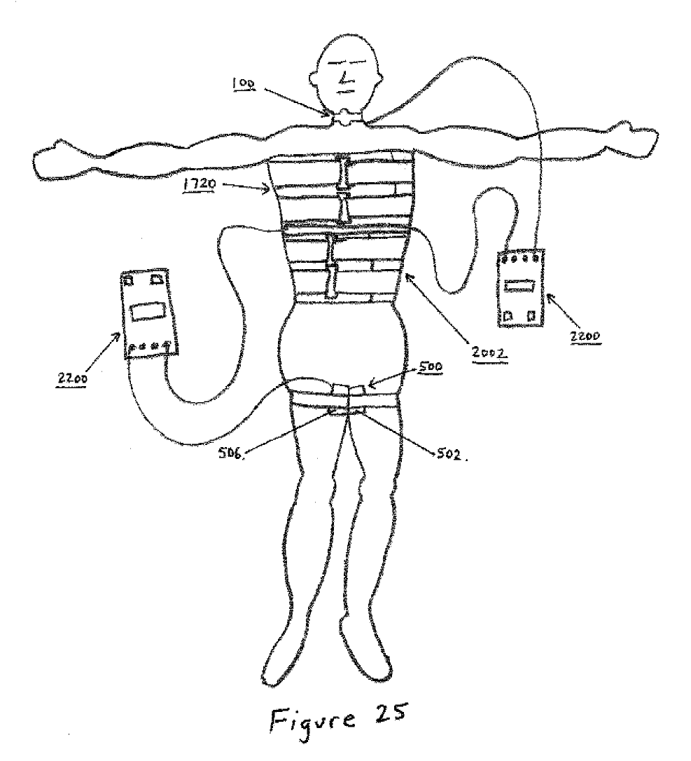

FIG. 24 illustrates an alternate apparatus and method to prevent or treat hypothermia. FIG. 24 illustrates that only the carotid, femoral and axillary blood vessels are warmed. The apparatus illustrated in FIG. 24 could be used to prevent or treat mild, moderate and severe hypothermia.

FIG. 25 illustrates a system and method similar to the system and method described in FIG. 23. FIG. 25 illustrates a patient laying supine with a torso warmer 1720 and a second torso warmer 2002 applying warmth to the chest wall and penetrating to heat the vascular lung tissue and axillary blood vessels. FIG. 25 illustrates an additional torso warmer 1720 and an additional second torso warmer 2002 to warm the abdomen. The neck collar 100 is applied loosely to the neck of the patient in order to provide warmth to the carotid blood vessels and trachea. The groin warming pad 500 provides warmth to the femoral blood vessels. The configuration described above could be used to prevent or treat mild and moderate hypothermia with important areas of the body being warmed. The extremities are left to be warmed at a slower rate reducing the risk of core temperature after drop.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1) A system for hypothermia warming of a user, comprising:
a first torso warming device to warm an upper torso of a user;
said first upper torso warming device including a first strap to connect said torso warming device to said user.

2) A system for hypothermia warming of a user as in claim 1, wherein said first torso warming device includes a central portion.

3) A system for hypothermia warming of a user as in claim 1, wherein said first upper torso warming device includes a first flap and a second flap to connect with said first strap around the circumference of the torso and fastened by a fastener.

4) A system for hypothermia warming of a user as in claim 1, wherein said system includes a second upper torso warming device to warm the lower torso of the user.

5) A system for hypothermia warming of a user as in claim 4, wherein said second upper torso warming device includes a second central portion.

6) A system for hypothermia warming of a user as in claim 4, wherein said second upper torso warming device includes a third flap and a fourth flap to connect with a second strap around the circumference of the lower torso and fastened by a fastener.

7) A system for hypothermia warming of a user as in claim 1, wherein said first strap includes a Velcro portion.

8) A system for hypothermia warming of a user as in claim 6, wherein said second strap includes a Velcro portion.

9) A system for hypothermia warming of a user as in claim 1, wherein said first upper torso warming device operates on DC current.

10) A system for hypothermia warming of a user as in claim 1, wherein said system includes a groin warming pad to warm only a portion of the groin of the user.

11) A system for hypothermia warming of a user as in claim 1, wherein said system includes a neck collar to warm the exterior portion of the neck of the user.

12) A system for hypothermia warming of a user as in claim 10, wherein said groin warming pad includes a groin strap.

13) A system for hypothermia warming of a user as in claim 10, wherein said groin warming pad operates on DC current.

14) A system for hypothermia warming of a user as in claim 11, wherein said neck collar includes a neck strap.

15) A system for hypothermia warming of a user as in claim 11, wherein said neck collar operates on DC current.

16) A system for hypothermia warming of a user as in claim 1, wherein said system includes an axillary warming pad to warm only a portion of a armpit of said user.
